(19) United States
(12) Patent Application Publication
(10) Pub. No.: US 2022/0381864 A1
(43) Pub. Date: Dec. 1, 2022

(54) BREATHING AND MOTION MONITORING METHOD FOR MRI SYSTEM, MRI SYSTEM AND METHOD, AND STORAGE MEDIUM

(71) Applicant: GE Precision Healthcare LLC, Wauwatosa, WI (US)

(72) Inventors: Yaan Ge, Beijing (CN); Liya Ma, Beijing (CN); Sheng Tong, Beijing (CN); Qingyu Dai, Beijing (CN); Kun Wang, Beijing (CN)

(21) Appl. No.: 17/738,219

(22) Filed: May 6, 2022

(30) Foreign Application Priority Data
May 31, 2021 (CN) 202110599033.2

(51) Int. Cl. G01R 33/567

(52) U.S. Cl. CPC G01R 33/5673

(57) ABSTRACT

The present application provides a breathing and motion monitoring method for an MRI system, an MRI system and method, and a storage medium. The MRI includes a scanner, a controller, and a signal processor. The scanner includes a radio-frequency transmit chain and a radio-frequency transmit coil, and an object under detection is positioned relative to the radio-frequency transmit coil. The controller is configured to control the scanner to perform a scanning sequence on the object under detection to acquire image data. The scanning sequence includes a radio-frequency excitation stage, a signal acquisition stage, and an idle stage. In the radio-frequency excitation stage, the radio-frequency transmit chain transmits a first radio-frequency pulse to the radio-frequency transmit coil. The signal processor is configured to acquire scattering parameters of the radio-frequency transmit coil in real time, wherein in the radio-frequency excitation stage, a first radio-frequency power signal detected on a line between the radio-frequency transmit chain and the radio-frequency transmit coil is acquired in real time, and the scattering parameters are acquired on the basis of the signal; and at least one of breathing information and motion information of the object under detection is acquired on the basis of the scattering parameters.

### CROSS REFERENCE

The present application claims priority and benefit of Chinese Patent Application No. 202110599033.2 filed on May 31, 2021, which is incorporated herein by reference in its entirety.

### FIELD OF THE INVENTION

The present invention relates to medical imaging technology, and more specifically, to a breathing and motion monitoring method for a magnetic resonance imaging system, a magnetic resonance imaging system, and a non-transitory computer-readable storage medium.

### BACKGROUND OF THE INVENTION

In some clinical applications of magnetic resonance imaging, in order to reduce breathing artifacts, an object under detection needs to hold breath when being scanned, or a breathing curve of the object under detection needs to be predicted by means of some techniques before scanning. In this way, scanning can be performed in a relatively smooth stage of the predicted breathing curve in a scanning and imaging process, such that an image with fewer artifacts can be acquired. The previous approach has high requirements on the object under detection, which increases the difficulty of scanning, while the latter approach increases scanning time, and the prediction accuracy thereof needs to be improved.

### BRIEF DESCRIPTION OF THE INVENTION

One aspect of an embodiment of the present invention provides a breathing and motion monitoring method for a magnetic resonance imaging system. The magnetic resonance imaging system comprises a scanner and a controller. The controller is configured to control the scanner to perform a scanning sequence on an object under detection to acquire image data of the object under detection. The scanner comprises a radio-frequency transmit chain and a radio-frequency transmit coil. The object under detection is positioned relative to the radio-frequency transmit coil. The scanning sequence comprises a radio-frequency excitation stage, a signal acquisition stage, and an idle stage between the radio-frequency excitation stage and the signal acquisition stage. The breathing and motion monitoring method comprises: acquiring scattering parameters in real time during the scanning sequence. The scanning sequence includes in the radio-frequency excitation stage, acquiring, in real time, a first radio-frequency power signal detected on a line between the radio-frequency transmit chain and the radio-frequency transmit coil, and acquiring the scattering parameters on the basis of the first radio-frequency power signal. The method further comprises acquiring at least one of breathing information and motion information of the object under detection on the basis of the scattering parameters acquired in real time.

In another aspect, acquiring the scattering parameters in real time further comprises: in the idle stage, controlling the radio-frequency transmit chain to transmit a second radio-frequency pulse to the radio-frequency transmit coil. During transmission of the second radio-frequency pulse, in real time, a second radio-frequency power signal detected on the line between the radio-frequency transmit chain and the radio-frequency transmit coil is acquired. Further, acquiring the scattering parameters includes acquiring the scattering parameters on the basis of the second radio-frequency power signal.

In another aspect, each of the frequency of the second radio-frequency pulse and the frequency of the first radio-frequency pulse is an operating frequency of the magnetic resonance imaging system. The first radio-frequency pulse has a first power capable of exciting the object under detection, and the second radio-frequency pulse has a second power incapable of exciting the object under detection.

In another aspect, the magnetic resonance imaging system further comprises a first additional radio-frequency transmit chain, and the step of acquiring scattering parameters in real time in the method further comprises: in the idle stage, controlling the first additional radio-frequency transmit chain to transmit a third radio-frequency pulse to the radio-frequency transmit coil. During transmission of the third radio-frequency pulse, in real time, a third radio-frequency power signal detected on a line between the first additional radio-frequency transmit chain and the radio-frequency transmit coil is acquired. Further, the step of acquiring scattering parameters includes acquiring the scattering parameters on the basis of the third radio-frequency power signal.

In another aspect, a frequency range of the third radio-frequency pulse deviates from an operating frequency range of the magnetic resonance imaging system. In another aspect, a power of the third radio-frequency pulse is in milliwatts or watts.

In another aspect, the magnetic resonance imaging system further comprises a second additional radio-frequency transmit chain. Moreover, in the method, the step of acquiring the scattering parameters in real time further comprises: in the signal acquisition stage, controlling the second additional radio-frequency transmit chain to transmit a fourth radio-frequency pulse to the radio-frequency transmit coil. During transmission of the fourth radio-frequency pulse, in real time, a fourth radio-frequency power signal detected on a line between the second additional radio-frequency transmit chain and the radio-frequency transmit coil is acquired. The step of acquiring the scattering parameters further comprises acquiring the scattering parameters on the basis of the fourth radio-frequency power signal.

In another aspect, a frequency range of the fourth radio-frequency pulse deviates from an operating frequency range of the magnetic resonance imaging system.

In another aspect, the breathing information of the object under detection is acquired, on the basis of a first filter, from the scattering parameters acquired in real time, and the motion information of the object under detection is acquired, on the basis of a second filter, from the scattering parameters acquired in real time.

In another aspect, an embodiment of the present invention further provides a magnetic resonance imaging method, comprising the breathing and motion monitoring method according to any of the above aspects, and further comprising: processing image data of the object under detection on the basis of at least one of the breathing information and the motion information of the object under detection.

In another aspect, an embodiment of the present invention further provides a computer-readable storage medium comprising a stored computer program, wherein the computer program, when being executed, implements the method according to any one of the above aspects.

In another aspect, an embodiment of the present invention further provides a magnetic resonance imaging system, comprising a scanner and a controller. The scanner includes a radio-frequency transmit chain and a radio-frequency transmit coil, an object under detection being positioned relative to the radio-frequency transmit coil. The controller is configured to control the scanner to perform a scanning sequence on the object under detection to acquire image data of the object under detection. The scanning sequence includes a radio-frequency excitation stage, a signal acquisition stage, and an idle stage between the radio-frequency excitation stage and the signal acquisition stage, wherein in the radio-frequency excitation stage, the radio-frequency transmit chain transmits a first radio-frequency pulse to the radio-frequency transmit coil. The magnetic resonance imaging system also includes a signal processor, configured to: acquire scattering parameters in real time during the scanning sequence which includes: in the radio-frequency excitation stage, acquiring, in real time, a first radio-frequency power signal detected on a line between the radio-frequency transmit chain and the radio-frequency transmit coil, and acquiring scattering parameters on the basis of the first radio-frequency power signal. The signal processor is further configured to acquire at least one of breathing information and motion information of the object under detection on the basis of the scattering parameters acquired in real time.

In another aspect, the controller is further configured to: in the idle stage, control the radio-frequency transmit chain to transmit a second radio-frequency pulse to the radio-frequency transmit coil. The signal processor is also configured to: during transmission of the second radio-frequency pulse, acquire, in real time, a second radio-frequency power signal detected on the line between the radio-frequency transmit chain and the radio-frequency transmit coil; and acquire the scattering parameters on the basis of the second radio-frequency power signal.

In another aspect, each of the frequency of the second radio-frequency pulse and the frequency of the first radio-frequency pulse is an operating frequency of the magnetic resonance imaging system, the first radio-frequency pulse has a first power capable of exciting the object under detection, and the second radio-frequency pulse has a second power incapable of exciting the object under detection.

In another aspect, the system further comprises a first additional radio-frequency transmit chain. The controller is further configured to: in the idle stage, control the first additional radio-frequency transmit chain to transmit a third radio-frequency pulse to the radio-frequency transmit coil. The signal processor is further configured to: during transmission of the third radio-frequency pulse, acquire, in real time, a third radio-frequency power signal detected on a line between the first additional radio-frequency transmit chain and the radio-frequency transmit coil; and acquire the scattering parameters on the basis of the third radio-frequency power signal.

In another aspect, a frequency range of the third radio-frequency pulse deviates from an operating frequency range of the magnetic resonance imaging system.

In another aspect, a power of the third radio-frequency pulse is in milliwatts or watts.

In another aspect, the system further comprises a second additional radio-frequency transmit chain. The controller is further configured to: in the signal acquisition stage, control the second additional radio-frequency transmit chain to transmit a fourth radio-frequency pulse to the radio-frequency transmit coil. The signal processor is also configured to: during transmission of the fourth radio-frequency pulse, acquire, in real time, a fourth radio-frequency power signal detected on a line between the second additional radio-frequency transmit chain and the radio-frequency transmit coil; and acquire the scattering parameters on the basis of the fourth radio-frequency power signal.

In another aspect, a frequency range of the fourth radio-frequency pulse deviates from an operating frequency range of the magnetic resonance imaging system.

In another aspect, the signal processor is configured to extract breathing information of the object under detection from the scattering parameters acquired in real time on the basis of a first filter, and the signal processor is configured to extract the motion information of the object under detection from the scattering parameters acquired in real time on the basis of a second filter.

In another aspect, the system further comprises an image data processor configured to process image data of the object under detection on the basis of at least one of the breathing information and the motion information of the object under detection. Other features and aspects will become apparent from the following detailed description, accompanying drawings, and claims.

### BRIEF DESCRIPTION OF THE DRAWINGS

The present invention can be better understood through the description of exemplary embodiments of the present invention in conjunction with the accompanying drawings, in which:

FIG. 1 is a schematic diagram of an MRI system according to some embodiments of the present invention;

FIG. 2 is a schematic diagram of an MRI system according to some other embodiments of the present invention;

FIG. 3 is a schematic diagram of an MRI system according to some other embodiments of the present invention;

FIG. 4 illustrates the scattering parameters acquired during transmission of a first radio-frequency pulse or a second radio-frequency pulse in some embodiments of the present invention;

FIG. 5 illustrates the breathing information acquired on the basis of the scattering parameters of FIG. 4;

FIG. 6 illustrates the motion information acquired on the basis of the scattering parameters of FIG. 4;

FIG. 7 illustrates the scattering parameters acquired during transmission of a third radio-frequency pulse or a fourth radio-frequency pulse in some embodiments of the present invention;

FIG. 8 illustrates the breathing information acquired on the basis of the scattering parameters of FIG. 7;

FIG. 9 illustrates the motion information acquired on the basis of the scattering parameters of FIG. 7;

FIG. 10 is a flowchart of a breathing and motion monitoring method for a magnetic resonance imaging system according to some embodiments of the present invention; and

FIG. 11 is a flowchart of a magnetic resonance imaging method according to some embodiments of the present invention.

### DETAILED DESCRIPTION

Specific implementations of the present invention will be described below. It should be noted that in the specific description of these embodiments, for the sake of brevity and conciseness, this specification may not describe all features of the actual implementations in detail. It should be understood that in the actual implementation process of any implementations, just as in the process of any engineering project or design project, a variety of specific decisions are often made to achieve specific goals of the developer and to meet system-related or business-related constraints, which may also vary from one implementation to another. Furthermore, it should also be understood that although efforts made in such development processes may be complex and tedious, for those of ordinary skill in the art related to the content disclosed in the present invention, some design, manufacture or production changes on the basis of the technical content disclosed in the present disclosure are only common technical means, and should not be construed as insufficient content of the present disclosure.

Unless defined otherwise, technical terms or scientific terms used in the claims and specification should have usual meanings understood by those of ordinary skill in the technical field to which the present invention belongs. The terms “first,” “second,” and similar terms used in the description and claims of the patent application of the present invention do not denote any order, quantity, or importance, but are merely intended to distinguish between different constituents. The terms “one” or “a/an” and similar terms do not denote a limitation of quantity, but rather the presence of at least one. The terms “include” or “comprise” and similar terms mean that an element or article in front of “include” or “comprise” encompass elements or articles and their equivalent elements listed after “include” or “comprise”, and do not exclude other elements or articles. The term “connect” or “connected” and similar words are not limited to physical or mechanical connections, and are not limited to direct or indirect connections.

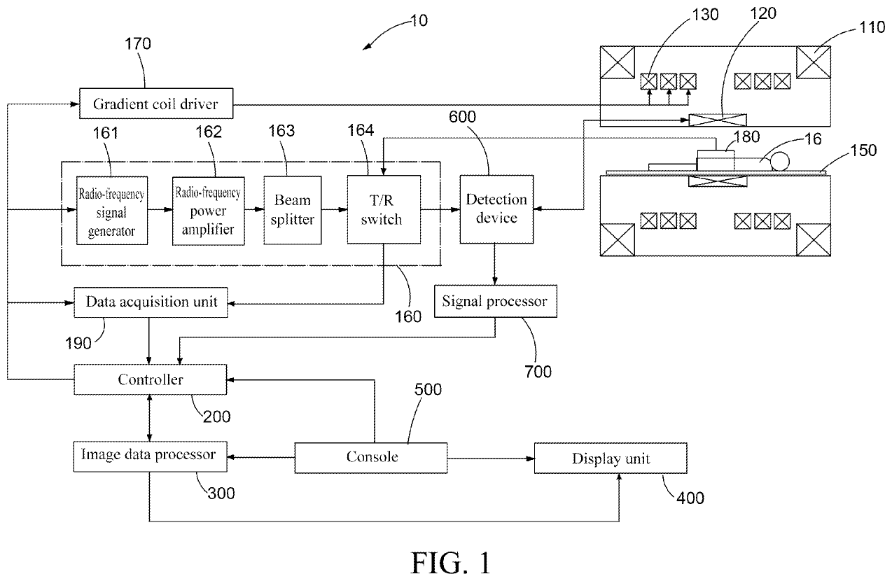

FIG. 1 is a schematic diagram of an MRI system 10 according to some embodiments of the present invention. As shown in FIG. 1, the MRI system 10 includes a scanner 100 and a controller 200.

The scanner 100 may be configured to acquire data of an object under detection 16. The controller 200 is coupled to the scanner 100 to control operation of the scanner 100, e.g., to control the scanner 100 to perform a scanning sequence on the object 16 to acquire image data of the object under detection 16.

Specifically, the controller 200 may send a sequence control signal to relevant components of the scanner 100 (including a radio-frequency generator and/or a gradient coil driver, etc., which will be described below) by means of a sequence generator (not shown in the figure), causing the scanner 100 to perform a preset scanning sequence.

Performing a magnetic resonance scan on the object 16 may include a positioning scan (3-plane scan) and a formal scan. One or more scanning sequences may be performed during the positioning scan and formal scan. During the positioning scan, at least one of a coronal positioning image, a sagittal positioning image, and a transverse section positioning image of the object may be acquired, and scan parameters of the formal scan, e.g., the scan range of the formal scan, are determined on the basis of this positioning image. Prior to performing one or more scanning sequences of the positioning scan or the formal scan, a pre-scan may be performed automatically or manually. During the pre-scan process, frequency adjustment may be performed to determine the Larmor frequency of proton resonance of the current scan on the basis of magnetic resonance signal feedback at different frequencies, and a radio-frequency transmit intensity adjustment may be made to determine the radio-frequency transmit power of the current scan on the basis of the magnetic resonance signal feedback at different radio-frequency transmit intensities.

Those skilled in the art would understand that the “scanning sequence” refers to a combination of pulses applied during magnetic resonance imaging scanning, and having specific power, amplitude, width, direction, and timing sequence (different clinical applications may include different pulse combinations). The pulses may typically include, for example, radio-frequency pulses and gradient pulses. The radio-frequency pulses may include, for example, a radio-frequency transmit pulse for exciting protons in the body to resonate. The gradient pulses may include, for example, a slice selection gradient pulse, a phase encoding gradient pulse, a frequency encoding gradient pulse, etc. Typically, a plurality of scanning sequences can be pre-set in the magnetic resonance imaging system, so that the sequence suitable for clinical detection requirements can be selected. The clinical detection requirements may include, for example, an imaging site, an imaging function, an imaging effect, and the like.

The scanner 100 usually includes an annular superconducting magnet defined in a housing. The annular superconducting magnet is installed in an vacuum container, and forms a cylindrical space.

In some embodiments, the scanner 100 may include a radio-frequency transmit coil 120 that may include a body coil disposed along an inner ring of the annular superconducting magnet.

In some embodiments, the scanner 100 includes a gradient coil assembly 130 disposed between an inner surface of a main magnet assembly 110 and an outer surface of the radio-frequency transmit coil 120.

Those skilled in the art would understand that the scanner 100 may further include a housing (not shown in the figure), and the main magnet assembly 110, the radio-frequency transmit coil 120, the gradient coil assembly 130, and some other components can be disposed in the housing.

The object under detection 16 is positioned relative to the radio-frequency transmit coil 120. Specifically, an inner ring of the radio-frequency transmit coil 120 and the housing define a scanning bore for accommodating the object under detection 16.

In some embodiments, the scanner 100 includes a bed 150 configured to hold the object under detection 16, and travels in a Z direction (generally the extension direction from head to foot when the object under detection is located in the scanning bore) to enter or exit the scanning bore in response to the control of the controller 200. For example, in one embodiment, an imaging volume of the object under detection 16 can be positioned in a central region of the scanning bore having uniform magnetic field intensity so as to facilitate scanning and imaging of the imaging volume of the object under detection 16.

In some embodiments, the main magnet assembly 110 may generate a main magnetic field in the Z direction, such as a main magnetic field B0. The MRI system 10 transmits, using the formed main magnetic field B0, a static magnetic pulse signal to the object under detection 16 placed in an imaging space, such that the progress of the protons within the object under detection 16 is ordered, and a longitudinal magnetization vector is generated.

In some embodiments, the scanner 100 includes a radio-frequency transmit chain 160 that may be configured to transmit a radio-frequency power signal (or radio-frequency pulses) to the radio-frequency transmit coil 120.

In some embodiments, the radio-frequency transmit chain includes a radio-frequency signal generator 161, a radio-frequency power amplifier 162, a beam splitter 163, and a transmit/receive (T/R) switch 164. The transmit/receive (T/R) switch 164 is connected to the radio-frequency transmit coil 120 to switch the radio-frequency transmit coil 120 to a radio-frequency power transmit mode or receive mode in response to the control signal of the controller 200.

The radio-frequency signal generator 161 is configured to generate the radio-frequency pulses in response to the sequence control signal of the controller 200. The radio-frequency pulses may include a radio-frequency excitation pulse. In a radio-frequency transmit mode, the radio-frequency excitation pulse is amplified by the radio-frequency power amplifier 162 (e.g., via the beam splitter 163 and the T/R switch 164) and then applied to the radio-frequency transmit coil 120 such that the radio-frequency transmit coil 120 emits to the object under detection 16 a radio-frequency magnetic field B1 that is orthogonal to the main magnetic field B0, to excite nuclei within the object under detection 16, and the longitudinal magnetization vector is transformed into a transverse magnetization vector.

The beam splitter 163 is configured to split a radio-frequency signal output by the radio-frequency power amplifier 162 into two orthogonal signals (with a phase difference of 90 degrees). One signal is transmitted via a first line (I-line) to the radio-frequency transmit coil 120, and the other signal is transmitted via a second line (Q-line) to the radio-frequency transmit coil 120.

After the radio-frequency excitation pulse ends, a free induction decay signal, i.e. a magnetic resonance signal that can be acquired, is generated during the process in which the transverse magnetization vector of the object under detection 16 gradually returns to zero.

In some embodiments, the scanner 100 includes a gradient coil driver 170 configured to provide, in response to the sequence control signal sent by the controller unit 200, a suitable power signal to the gradient coil assembly 130, so that the gradient coil assembly 130 forms a magnetic field gradient in the imaging space, so as to provide three-dimensional positional information for the magnetic resonance signal described above.

Specifically, the gradient coil assembly 130 may include gradient coils in three directions. Each of the gradient coils in three directions generates a gradient magnetic field inclined in one of three spatial axes (e.g., X-axis, Y-axis, and Z-axis) perpendicular to one another, and generates a gradient field in each of a slice selection direction, a phase encoding direction, and a frequency encoding direction according to imaging conditions. Specifically, the gradient coil assembly 130 applies a gradient field in the slice selection direction of the object under detection 16 so as to select a radio-frequency excited slice. The gradient coil assembly 130 also applies a gradient field in the phase encoding direction of the object under detection 16 so as to perform phase encoding on the magnetic resonance signal of the excited slice. The gradient coil assembly 130 then applies a gradient field in the frequency encoding direction of the object under detection 16 so as to perform frequency encoding on the magnetic resonance signal of the excited slice.

The magnetic resonance signal having positional information can be received by the radio-frequency receive coil. For example, the controller 200 may control the transmit/receive (T/R) switch 164 to switch the radio-frequency transmit coil 120 to the receive mode and control the radio-frequency transmit coil 120 in the receive mode to receive the magnetic resonance signal from a particular coil channel.

The scanner 100 may further include a surface receive coil 180. The surface receive coil 180 is typically disposed proximate to a scan site (a region of interest) of the object under detection 16 (for example, overlaid or laid on a body surface of the object under detection 16). The surface receive coil 180 may also be configured to receive the magnetic resonance signal from the object 16. For example, the controller 200 may select a coil channel of the surface receive coil 180 for receiving the magnetic resonance signal.

In some embodiments, the scanner 100 may further include a data acquisition unit 190 configured to acquire the magnetic resonance signal received by the surface receive coil 180 or the radio-frequency transmit coil 120 in the receive mode. The data acquisition unit 190 may include, for example, a radio-frequency pre-amplifier (not shown), a phase detector (not shown), and an analog/digital converter (not shown). The radio-frequency pre-amplifier is configured to amplify the magnetic resonance signal received by the surface receive coil 180 or the radio-frequency transmit coil 120, the phase detector is configured to perform phase detection on the amplified magnetic resonance signal, and the analog/digital converter is configured to convert the magnetic resonance signal that has undergo phase detection from an analog signal into a digital signal.

In some embodiments, the data acquisition unit 190 is further configured to store the digitized magnetic resonance signal (or echo) in a K space in response to the control signal of the controller 200. The K-space is a space to which raw data of magnetic resonance signals carrying spatial orientation encoding information is populated.

In some embodiments, the MRI system 10 further includes an image data processor 300. The raw data may be processed by the image data processor 300 such that a desired medical magnetic resonance image is obtained. This processing may include, for example, signal pre-processing, image reconstruction, and post processing, etc.

For example, the image data processor 300 may include an image reconstruction unit configured to perform inverse Fourier transform on the data stored in the K space to reconstruct a three-dimensional image or a two-dimensional slice image of the imaging volume of the object 16.

In some embodiments, the MRI system 10 may further include a display unit 400 configured to display an operating interface as well as various data, images or parameters generated during data acquisition and processing.

In some embodiments, the MRI system 10 includes an operating console 500, which may include a user input apparatus, such as a keyboard and mouse, etc. The controller 200 may communicate with the scanner 100, image data processor 300, display unit 400, etc., in response to a control command generated by a user on the basis of the operating console 500 or an operating panel/key or the like disposed on a main magnet housing. The control command may include, for example, a scanning protocol, a parameter, etc. selected manually or automatically. The scanning protocol may include the scanning sequence described above.

In an embodiment of the present invention, a signal processor 700 may be further included, and can configure parameters or acquire required information on the basis of the feedback/detected signal, e.g., acquire the scattering parameters in real time, and acquire at least one of the breathing information and motion information of the object in real time on the basis of the scattering parameters.

In some embodiments, the signal processor 700 may be integrated with the controller 200 or (e.g., in the form of a module) as part of the controller 200. The controller 200, the image data processor 300, and the signal processor 700 may be separately or collectively include a computer processor and a storage medium on which predetermined data processing programs to be executed by the computer processor are stored. For example, programs for performing scanning, signal pre-processing, image reconstruction and image post-processing are stored on the storage medium, and programs for implementing the breathing and motion monitoring method and the magnetic resonance imaging method according to the embodiments of the present invention may also be stored on the storage medium. The storage medium may include, for example, a ROM, a floppy disk, a hard disk, an optical disk, a magneto-optical disk, a CD-ROM, or a non-volatile memory card.

An embodiment of the present invention may further provide a non-transitory computer-readable storage medium including a stored instruction set and/or computer program. The instruction set and/or computer program, when being executed, implements the breathing and motion monitoring method or the magnetic resonance imaging method according to the embodiment of the present invention. The method will be described in detail below.

As used herein, the term “computer” may include any processor-based or microprocessor-based system that includes a system using a microcontroller, a reduced instruction set computer (RISC), an application specific integrated circuit (ASIC), a logic circuit, and any other circuit or processor capable of performing the functions described herein. The examples above are exemplary only and are not intended to limit the definition and/or meaning of the term “computer” in any way.

Instructions in the instruction set may be combined into one instruction for execution, and any instruction may also be split into a plurality of instructions for execution. Furthermore, the instructions are not limited to be executed according to the instruction execution sequence described above.

The instruction set may include various commands used to instruct the computer serving as a processing machine or the processor to perform specific operations, e.g., methods and processes of various embodiments. The instruction set may be in the form of a software program that may form part of one or more tangible, non-transitory computer readable media. The software may be in various forms of, e.g., system software or application software. Furthermore, the software may be in the form of a standalone program or a collection of modules, a program module within a larger program, or part of a program module. The software may also include modular programming in the form of object-oriented programming. Processing of the input data by the processing machine may be in response to an operator command, or in response to a previous processing result, or in response to a request made by another processing machine.

The controller 200, the image data processor 300 and the signal processor 700 may be configured and/or arranged to be used in different manners. For example, in some implementations, a single unit may be used. In other implementations, a plurality of (control or processing) units are configured to operate together (e.g., on the basis of a distributed processing configuration) or separately. Each unit is configured to process a particular aspect and/or function, and/or to process data configured to generate a model for a particular MRI system alone. In some implementations, the controller 200, the image data processor 300, and the signal processor 700 can be local (e.g., within the same facility and/or same local network as one or more systems). In other implementations, the controller 200, the image data processor 300, and the signal processor 700 can be remote, and thus are only accessible via a remote connection (e.g., via the Internet or other available remote access technologies). In particular implementations, the controller 200, the image data processor 300, and the signal processor 700 may be configured in a cloud-like manner, and may be accessed and/or used in a manner substantially similar to the manner in which other cloud-based systems are accessed and/or used.

The MRI system 10 is only described as an example, and in other embodiments, the MRI system 10 may have various variations, as long as image data can be acquired from the object under detection.

In an embodiment of the present invention, the scanning sequence may include a radio-frequency excitation stage and a signal acquisition stage. The radio-frequency excitation stage may include: transmitting a first radio-frequency pulse to the radio-frequency transmit coil by means of the radio-frequency transmit chain, where the first radio-frequency pulse may be a radio-frequency excitation pulse, and has a first frequency and a first power capable of exciting the object under detection. For example, the first frequency is the operating frequency of the MRI system, and the first power is a radio-frequency power in kilowatts.

The signal acquisition stage may include: receiving the magnetic resonance signal by means of the selected coil channel in an array of surface receive coils, or switching the radio-frequency transmit coil to the receive mode and receiving the magnetic resonance signal by means of the selected coil channel therein. In some embodiments, the frequency encoding gradient pulse is applied in the signal acquisition stage. Between the radio-frequency excitation stage and the signal acquisition stage, an idle stage is further included, where sequence pulses with other functions may be applied, e.g., a radio-frequency refocusing pulse, a phase encoding gradient pulse, an inversion recovery pulse, etc., which will not be enumerated herein.

In some clinical applications (e.g., abdomen and chest detection), in order to reduce image artifacts caused by breathing, the object under detection needs to hold breath when the scanning sequence is performed on the object under detection, or a navigation techniques are used, i.e., a breathing motion curve of the object under detection is predicted by low resolution imaging, and the scanning sequence is performed in a relatively smooth stage of the predicted breathing curve, so as to obtain a desired high resolution image. In an embodiment of the present invention, the motion information and breathing information of the object under detection may be acquired in real time during the scanning sequence, so as to correspond the acquired raw image data with the motion and breathing information, so that the more desirable raw data (e.g., data acquired when the object under detection is motionless and/or breathing stably) can be selected during image reconstruction.

When the first radio-frequency pulse is transmitted to the radio-frequency transmit coil by means of the radio-frequency transmit chain, a first radio-frequency power signal can be detected on a line between the radio-frequency transmit chain and the radio-frequency transmit coil, and includes a forward signal and a reverse signal. For example, a front end of the radio-frequency transmit chain (e.g., on a transmission line closer to the radio-frequency transmit coil) has the forward signal transmitted to the radio-frequency transmit coil 120 and the reverse signal reflected from the radio-frequency transmit coil 120, and the scattering parameters (S-parameters for short) of the radio-frequency transmit coil may be acquired on the basis of the reverse signal and the forward signal. The present invention assumes and verifies that when the object under detection 16 has a periodic (or physiological) motion (e.g., breathing) or aperiodic (or active) motion (e.g., movement of the body or body part), the S-parameters correspondingly undergo periodic or aperiodic changes, and such changes can reflect breathing characteristics and motion characteristics of the object under detection. At least one of the breathing information and motion information of the object under detection may be acquired by acquiring the S-parameters in real time.

In an embodiment of the present invention, the signal processor 700 may be configured to acquire the scattering parameters in real time when the scanning sequence is executed, including: in the radio frequency excitation stage, acquiring, in real time, the first radio-frequency power signal detected on the line between the radio-frequency transmit chain 130 and the radio-frequency transmit coil 120, and acquiring the scattering parameters on the basis of the first radio frequency power signal.

To avoid resource redundancy, the first radio-frequency power signal may be a radio-frequency excitation signal (i.e., the first radio-frequency pulse) itself.

Specifically, the first radio-frequency power signal may include a first forward power signal and a first reverse power signal, where the first forward power signal represents a power signal detected on the line and transmitted from the radio-frequency transmit chain to the radio-frequency transmit coil, and the first reverse power signal represents the power signal detected on the line and reflected from the radio-frequency transmit coil back to the radio-frequency transmit chain.

In order to more accurately measure the scattering parameters of the radio-frequency transmit coil, the first radio-frequency power signal can be detected at a position as close as possible to the radio-frequency transmit coil in the radio-frequency transmit chain. For example, the first radio-frequency power signal may be detected at a front end (the end closer to the radio-frequency transmit coil 120) of a line between the transmit/receive (T/R) switch 164 and the radio-frequency transmit coil 120.

Those skilled in the art would understand that the first radio-frequency power signal may be acquired by a detection device 600 disposed at a detection position. In some embodiments, the detection device 600 may be disposed on the I line and Q line respectively, and data acquired on the basis of the detection device 600 on the I line and the Q line may be fused to obtain scattering information, or only the detection device 600 on the I line or the Q line is used to detect the first radio frequency power signal. The detection device 600 detects and feeds back the first power signal in response to the control signal of the signal processor 700. In one embodiment, the detection device includes a directional coupler.

Specifically, the detection device, in response to the signal processor 700, may acquire the scattering parameters, e.g., acquire the scattering parameters of the radio-frequency transmit coil, on the basis of the ratio of the first reverse power signal to the first forward power signal.

Furthermore, the signal processor 700 is further configured to acquire at least one of the breathing information and motion information of the object under detection on the basis of the scattering parameters acquired in real time.

For example, the signal processor 700 may acquire a time-varying curve of the scattering parameters continuously and in real time, and apply a suitable filter to filter the acquired scattering parameters, so as to extract the breathing information and/or motion information of the object under detection. The filter may be a low pass filter.

In one embodiment, the signal processor 700 acquires the required breathing information and motion information by setting frequency selection of the filter, e.g., applying the first filter to filter the scattering parameters to acquire the breathing information, and applying the second filter to filter the scattering parameters to acquire the motion information.

In the radio-frequency excitation stage during the scanning sequence, since the radio-frequency transmit chain needs to transmit the radio-frequency excitation signal to the radio-frequency transmit coil to excite the nuclei of the tissue under detection to generate resonance, the scattering parameters can be acquired by monitoring the forward and reverse signals of the signal in real time, without needing to dispose an additional radio-frequency signal transmit source, thereby reducing hardware costs.

Typically, the time required to perform one repetition time (TR) of the scanning sequence is short. Thus, acquiring only the motion and breathing information of the object under detection in the radio-frequency excitation stage is sufficient to help obtain less motion or breathing artifacts. However, in order to meet special or higher requirements, at least one of the motion information and breathing information of the object may also be monitored in real time in more time periods.

In a conventional scanning method, after the radio-frequency excitation stage ends, the radio-frequency transmit chain no longer transmits the radio-frequency signal. In an embodiment of the present invention, in order to further acquire the breathing and motion information in the idle stage, and to improve the accuracy and persistence of the information, after radio-frequency excitation ends, the controller 200 controls the radio-frequency transmit chain to continue to transmit a second radio-frequency pulse to the radio-frequency transmit coil. Also, during transmission of the second radio-frequency pulse, the signal processor 700 is further configured to acquire, in real time, a second radio-frequency power signal detected on the line between the radio-frequency transmit chain and the radio-frequency transmit coil, and to acquire the scattering parameters on the basis of the second power signal.

The second radio-frequency power signal may be detected at the same detection position or using the same detection device.

Similar to the first radio-frequency power signal, the second radio-frequency power signal may include a second forward power signal and a second reverse power signal. The signal processor 700 may acquire the scattering parameters on the basis of the ratio of the second reverse power signal to the second forward power signal.

In the embodiment described above, since the radio-frequency transmit chain is idle in the idle stage, the radio-frequency transmit chain may be used to transmit the second radio-frequency pulse to generate the second radio-frequency power signal that can be detected. Furthermore, in order to avoid exciting the object under detection 16 in the idle stage, the second radio-frequency pulse has a second power incapable of exciting the object under detection. In addition, in order to reduce energy consumption, the second power may be in watts or milliwatts.

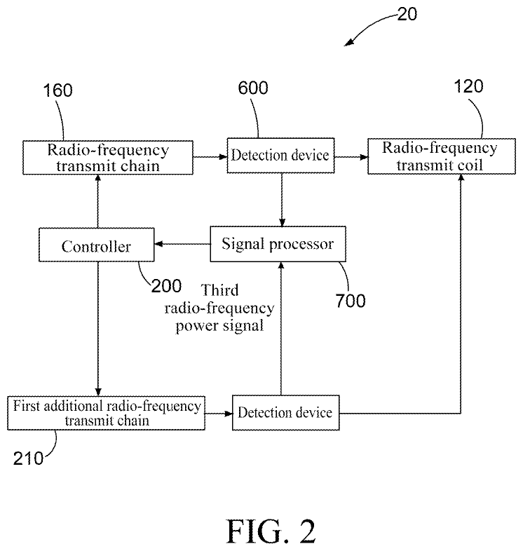

FIG. 2 is a schematic diagram of an MRI system 20 according to another embodiment. The MRI system 20 is similar to the MRI system 10, and differs in that the MRI system 20 includes a first additional radio-frequency transmit chain 210. The first additional radio-frequency transmit chain 210 includes a radio-frequency source that is additionally disposed and independent of the radio frequency transmit chain 130.

In an idle stage of a scanning sequence performed by the MRI system 20, the controller 200 is configured to control the first additional radio-frequency transmit chain 210 to transmit a third radio-frequency pulse to the radio-frequency transmit coil 120.

During transmission of the third radio-frequency pulse, the signal processor 700 further acquires, in real time, a third radio-frequency power signal detected on a line between the first additional radio-frequency transmit chain 210 and the radio-frequency transmit coil 120, and acquires scattering parameters on the basis of the third radio-frequency power signal.

Specifically, an additional detection device may be disposed at a front end (the end closer to the radio-frequency transmit coil 120) of the line between the first additional radio-frequency transmit chain 210 and the radio-frequency transmit coil 120 to detect the third radio-frequency power signal.

The third radio-frequency power signal may include a third forward power signal transmitted from the first additional radio-frequency transmit chain 210 to the radio-frequency transmit coil 120, and a third reverse power signal transmitted from the radio-frequency transmit coil 120 back to the first additional radio-frequency transmit chain 210. The signal processor 700 may acquire the scattering parameters on the basis of the ratio of the third reverse power signal to the third forward power signal.

Likewise, in order to avoid exciting the object under detection 16 in the idle stage, the third radio-frequency pulse has a third power incapable of exciting the object under detection, and in order to reduce energy consumption, the third power may be in watts or milliwatts. Thus, the first additional radio-frequency transmit chain may include a radio-frequency source that only emits low-power signals, which is also beneficial for reducing the hardware space and costs.

Due to the use of the additional radio-frequency source, the frequency of the third radio-frequency pulse may be different from the operating frequency of the MRI system 20, and the frequency range of the third radio-frequency pulse may deviate from the operating frequency range of the MRI system (greater than or less than the operating frequency). Specifically, the frequency of the third radio-frequency pulse may be configured to a value incapable of exciting the object under detection 16.

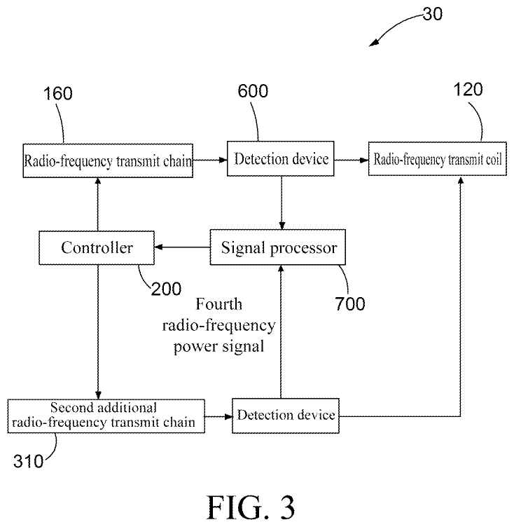

FIG. 3 is a schematic diagram of an MRI system 30 according to some other embodiments of the present invention. The MRI system 30 is similar to the MRI system 10, and differs in that the MRI system 30 includes a second additional radio-frequency transmit chain 310. The second additional radio-frequency transmit chain 310 includes a radio-frequency source that is additionally disposed and independent of the radio-frequency transmit chain 130 (for example, it may also be the first additional radio-frequency transmit chain 210).

In a signal acquisition stage of a scanning sequence performed by the MRI system 30, the controller 200 is configured to control the second additional radio-frequency transmit chain 310 to transmit a fourth radio-frequency pulse to the radio-frequency transmit coil 120.

During transmission of the fourth radio-frequency pulse, the signal processor 700 further acquires, in real time, a fourth radio-frequency power signal detected on a line between the second additional radio-frequency transmit chain 310 and the radio-frequency transmit coil 120, and acquires scattering parameters on the basis of the fourth radio-frequency power signal.

Specifically, an additional detection device may be disposed at a front end (the end closer to the radio-frequency transmit coil 120) of the line between the second additional radio-frequency transmit chain 310 and the radio-frequency transmit coil 120 to detect the fourth radio-frequency power signal.

The fourth radio-frequency power signal may include a fourth forward power signal transmitted from the second additional radio-frequency transmit chain 310 to the radio-frequency transmit coil 120, and a fourth reverse power signal reflected from the radio-frequency transmit coil 120 back to the second additional radio-frequency transmit chain 310. The signal processor 700 may acquire the scattering parameters on the basis of the ratio of the fourth reverse power signal to the fourth forward power signal.

In order to avoid exciting the object under detection 16 in the signal acquisition stage, the fourth radio-frequency pulse has a fourth power (which may be the same as the third power) incapable of exciting the object under detection, and in order to reduce energy consumption, the fourth power may be in watts or milliwatts. Thus, the second additional radio-frequency transmit chain 310 may include a radio-frequency source that only emits low-power signals, which is also beneficial for reducing the hardware space and costs.

Due to the use of the additional radio-frequency source, the frequency of the fourth radio-frequency pulse may be different from the operating frequency of the MRI system 30, and the frequency range of the fourth radio-frequency pulse may deviate from the operating frequency range of the MRI system 30 (greater than or less than the operating frequency). Specifically, the frequency of the fourth radio-frequency pulse may be configured to a value incapable of exciting the object under detection 16.

In an embodiment of the present invention, the signal processor 700 is configured to acquire a radio-frequency power signal (including one or more of the first to fourth radio-frequency power signals) detected during a peak period of a transmit radio-frequency pulse (e.g., one or more of the first to fourth radio-frequency transmit pulses), and to acquire scattering parameters of the radio-frequency transmit coil 120 on the basis of the radio-frequency power signal acquired during the peak period.

The peak period may be a continuous period of time having a pulse peak value, for example, within 50 microseconds in which higher pulse values (including the peak value) are obtained.

In other implementations, in the signal acquisition stage of the scanning sequence performed by the MRI system 30, it is possible to not dispose the additional radio-frequency transmit chain. Instead, when the radio-frequency transmit coil in the receive mode receives a magnetic resonance signal, a fifth reverse power signal transmitted from the human body to the radio-frequency transmit coil is detected at a receiving end of the radio-frequency transmit coil. The signal processor 700 may acquire scattering parameters on the basis of the ratio of the fifth reverse power signal to the first forward power signal, and acquire at least one of the breathing information and motion information of the object under detection 16 on the basis of the scattering parameters.

Alternatively, in the signal acquisition stage of the scanning sequence performed by the MRI system 30, when a magnetic resonance signal is received using the surface receive coil 180, a sixth reverse power signal transmitted from the human body to the surface receive coil 180 may be detected at a receiving end of the surface receive coil 180. The signal processor 700 may acquire scattering parameters on the basis of the ratio of the sixth reverse power signal to the first forward power signal, and acquire at least one of the breathing information and motion information of the object under detection 16 on the basis of the scattering parameters.

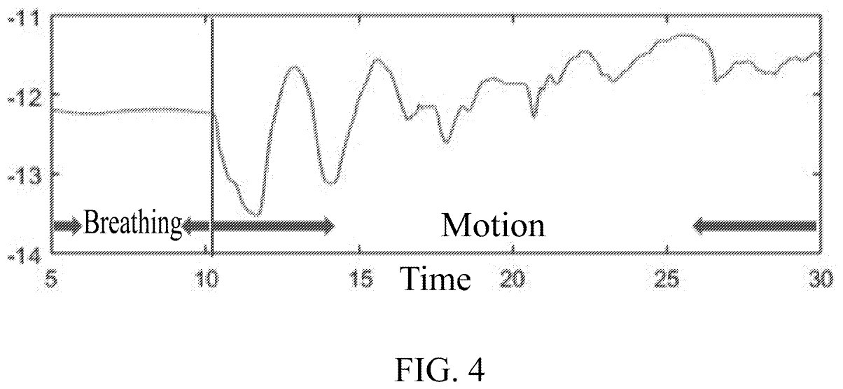

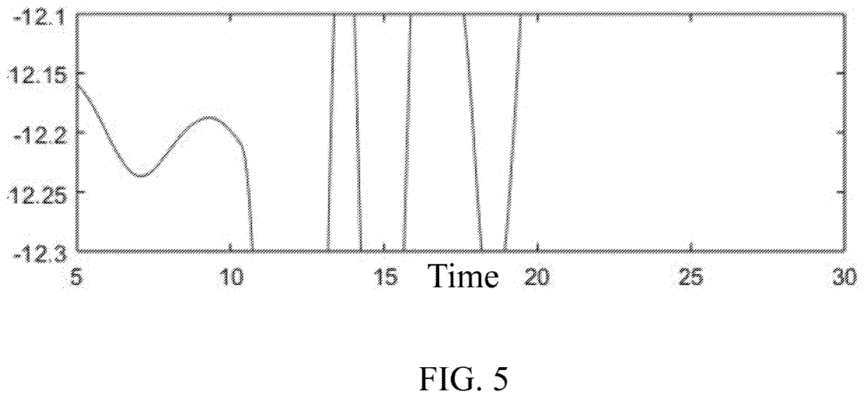

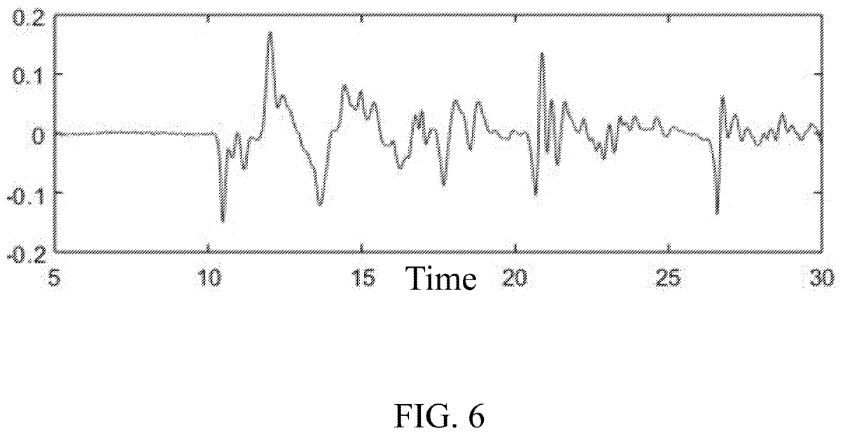

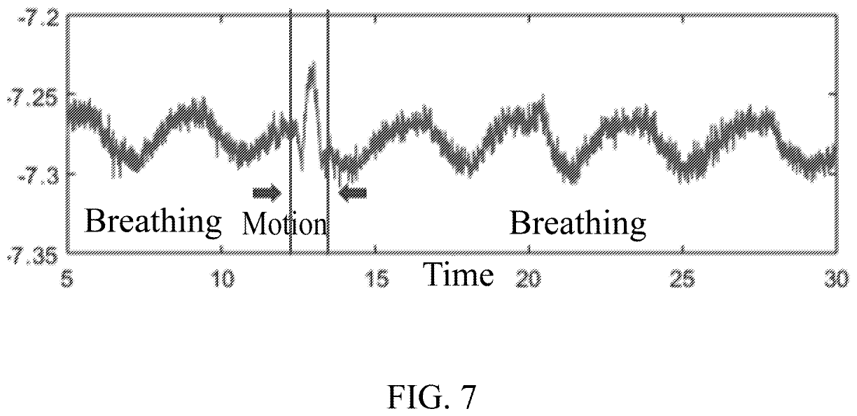

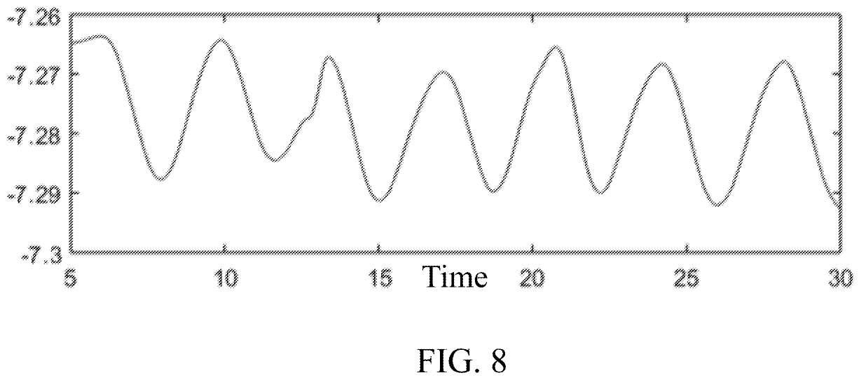

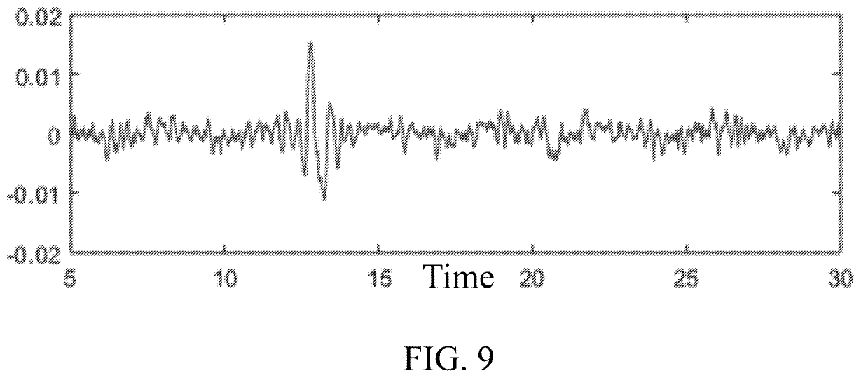

FIG. 4 illustrates the scattering parameters acquired during transmission of the first radio-frequency pulse or second radio-frequency pulse (the frequency thereof is the operating frequency of the MRI system), where the scattering parameters include scattering parameters acquired in a stable breathing stage and a motion stage of the object under detection. FIG. 5 illustrates the breathing information obtained on the basis of the scattering parameters of FIG. 4. FIG. 6 illustrates the motion information obtained on the basis of the scattering parameters of FIG. 4. FIG. 7 illustrates the scattering parameters acquired during transmission of the third radio-frequency pulse or fourth radio-frequency pulse (the frequency thereof deviates from the operating frequency), where the scattering parameters also include the scattering parameters acquired in the stable breathing stage and the motion stage. FIG. 8 illustrates the breathing information obtained on the basis of the scattering parameters of FIG. 7. FIG. 9 illustrates the motion information obtained on the basis of the scattering parameters of FIG. 7.

As described above, the image data processor 300 is configured to perform pre-processing, image reconstruction, post-processing, etc., on an acquired magnetic resonance signal. In an embodiment of the present invention, the image data processor is configured to process image data of the object under detection on the basis of at least one of the breathing information and motion information of the object under detection. For example, from FIGS. 5, 6, 8, and 9, it can be determined in which period the object under detection breathes stably, in which period breathing is not stable, in which period no motion occurs, in which period motion occurs, and so on. On the basis of the determined information, when the image data processor 300 performs image data processing on the acquired magnetic resonance signal, the processor may choose to retain suitable raw data (e.g., image data generated in the period in which breathing is stable and no motion occurs), and discard abnormal raw data (e.g., image data generated when breathing is not stable or motion occurs).

Reconstructed images obtained on the basis of the retained raw data have less breathing and motion artifacts. There is also no need to spend time predicting the breathing information of the object under detection before scanning the object, and no need to request the object under detection to hold breath at a specific time of scanning, thereby reducing scanning time and scanning difficulty.

It is verified by experiments that the breathing information and motion information obtained on the basis of an embodiment of the present invention are consistent with actual breathing and motion of the object under detection. Specifically, the breathing curve and the motion curve generated on the basis of the scattering information are consistent with reality when the object under detection inhales, exhales, holds breath and moves in the experiments. The breathing curve and the motion curve generated on the basis of the scattering information are consistent with reality when the objects under detection of different features (including body size, gender, age, etc.) breath deeply and freely in the experiments, respectively. When monitoring the breathing information of the object on the basis of a Bluetooth sensor and acquiring the breathing information on the basis of the scattering information are performed simultaneously, the consistency between the two operations is relatively high. The breathing information and the motion information acquired by starting the surface receive coil covering the abdomen or chest of the object under detection 16 are more consistent with the breathing information and the motion information acquired without using the surface receive coil, indicating that the use of the surface receive coil does not affect the accuracy of the breathing information and motion information.

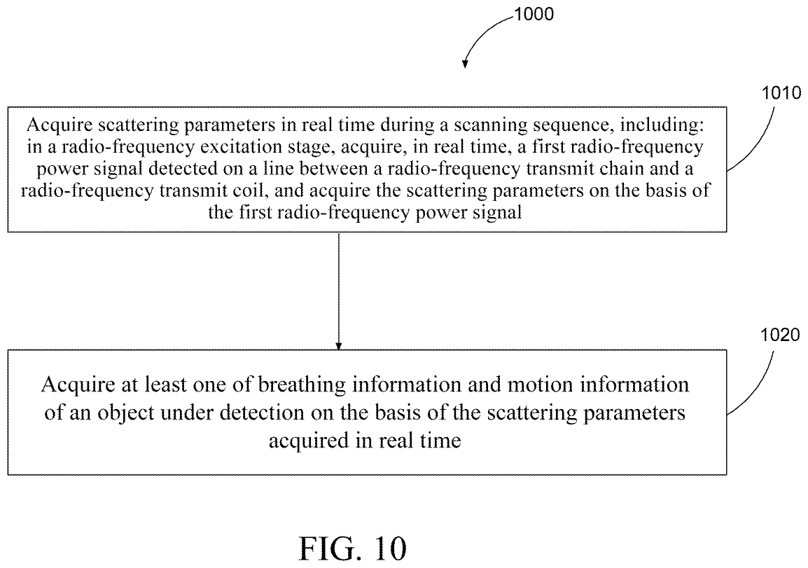

FIG. 10 is a flowchart 1000 of a breathing and motion monitoring method for a magnetic resonance imaging system according to some embodiments of the present invention. The magnetic resonance imaging system may include the magnetic resonance imaging system 10, 20, or 30 in the embodiments above. For example, the magnetic resonance imaging system includes a scanner and a controller. The controller is configured to control the scanner to perform a scanning sequence on an object under detection to acquire image data of the object under detection. The scanner includes a radio-frequency transmit chain and a radio-frequency transmit coil, and the object under detection is positioned relative to the radio-frequency transmit coil. The scanning sequence includes a radio-frequency excitation stage, a signal acquisition stage, and an idle stage between the radio-frequency excitation stage and the signal acquisition stage. As shown in FIG. 10, the method 1000 includes step 1010 and step 1020.

In step 1010, scattering parameters are acquired in real time during the scanning sequence, the step including: in a radio-frequency excitation stage, acquiring, in real time, a first radio-frequency power signal detected on a line between the radio-frequency transmit chain and the radio-frequency transmit coil, and acquiring the scattering parameters on the basis of the first radio-frequency power signal.

In step 1020, at least one of breathing information and motion information of the object under detection are acquired on the basis of the scattering parameters acquired in real time.

In some embodiments, step 1010 further includes: in the idle stage, controlling the radio-frequency transmit chain to transmit a second radio-frequency pulse to the radio-frequency transmit coil. During transmission of the second radio-frequency pulse, in real time, a second radio-frequency power signal detected on the line between the radio-frequency transmit chain and the radio-frequency transmit coil is acquired. The step 110 also includes acquiring the scattering parameters on the basis of the second radio-frequency power signal.

In some embodiments, each of the frequency of the second radio-frequency pulse and the frequency of the first radio-frequency pulse is an operating frequency of the magnetic resonance imaging system. The first radio-frequency pulse has a first power capable of exciting the object under detection, and the second radio-frequency pulse has a second power incapable of exciting the object under detection.

In some embodiments, the magnetic resonance imaging system further includes a first additional radio-frequency transmit chain, and step 1010 further includes: in the idle stage, controlling the first additional radio-frequency transmit chain to transmit a third radio-frequency pulse to the radio-frequency transmit coil. During transmission of the third radio-frequency pulse, in real time, a third radio-frequency power signal detected on a line between the first additional radio-frequency transmit chain and the radio-frequency transmit coil is acquired. The step 1010 also includes acquiring the scattering parameters on the basis of the third radio-frequency power signal.

In some embodiments, the frequency range of the third radio-frequency pulse deviates from the operating frequency range of the magnetic resonance imaging system.

In some embodiments, a power of the third radio-frequency pulse is in milliwatts or watts.

In some embodiments, the magnetic resonance imaging system further includes a second additional radio-frequency transmit chain, and step 1010 further includes: in the signal acquisition stage, controlling the second additional radio-frequency transmit chain to transmit a fourth radio-frequency pulse to the radio-frequency transmit coil. During transmission of the fourth radio-frequency pulse, in real time, a fourth radio-frequency power signal detected on a line between the second additional radio-frequency transmit chain and the radio-frequency transmit coil is acquired. The step 1010 also includes acquiring the scattering parameters on the basis of the fourth radio-frequency power signal.

In some embodiments, the frequency range of the fourth radio-frequency pulse deviates from the operating frequency range of the magnetic resonance imaging system.

In some embodiments, in step 1020, the breathing information of the object under detection is acquired, on the basis of a first filter, from the scattering parameters acquired in real time, and the motion information of the object under detection is acquired, on the basis of a second filter, from the scattering parameters acquired in real time.

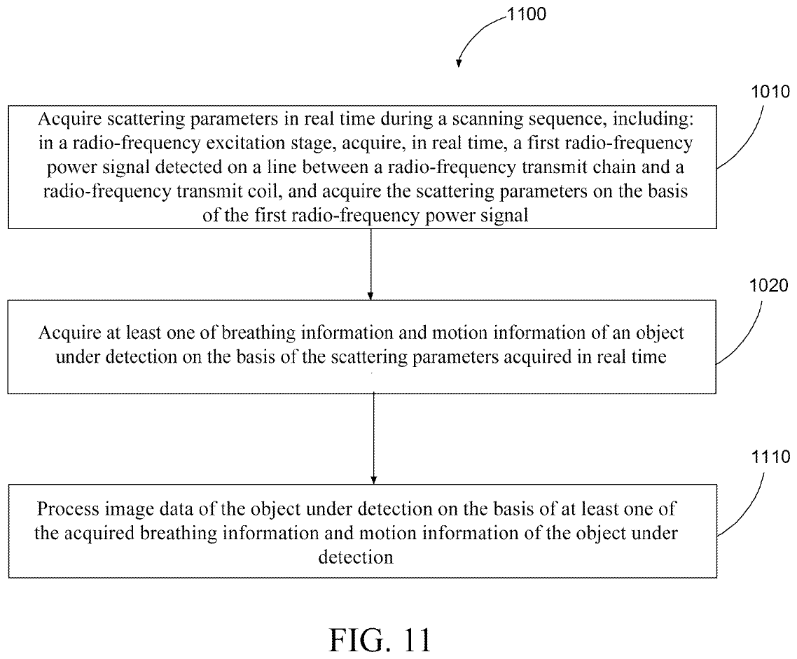

FIG. 11 is a flowchart 1100 of a magnetic resonance imaging method according to some embodiments of the present invention, which includes the breathing and motion monitoring method according to any of the above embodiments. The magnetic resonance imaging method 1100 further includes step 1110: processing image data of the object under detection on the basis of at least one of the acquired breathing information and motion information of the object under detection.

Some exemplary embodiments have been described above, however, it should be understood that various modifications may be made. For example, suitable results can be achieved if the described techniques are performed in different orders and/or if components in the described systems, architectures, devices, or circuits are combined in different ways and/or replaced or supplemented by additional components or equivalents thereof. Accordingly, other implementations also fall within the scope of the claims.

1. A breathing and motion monitoring method for a magnetic resonance imaging system, the magnetic resonance imaging system including a scanner and a controller, the controller being configured to control the scanner to perform a scanning sequence on an object under detection to acquire image data of the object under detection, the scanner comprising a radio-frequency transmit chain and a radio-frequency transmit coil, the object under detection being positioned relative to the radio-frequency transmit coil, the scanning sequence comprising a radio-frequency excitation stage, a signal acquisition stage, and an idle stage between the radio-frequency excitation stage and the signal acquisition stage, and the method comprising:
acquiring scattering parameters in real time during the scanning sequence, comprising: in the radio-frequency excitation stage, acquiring, in real time, a first radio-frequency power signal detected on a line between the radio-frequency transmit chain and the radio-frequency transmit coil, and acquiring the scattering parameters on the basis of the first radio-frequency power signal; and
acquiring at least one of breathing information and motion information of the object under detection on the basis of the scattering parameters acquired in real time.

2. The method according to claim 1, wherein acquiring the scattering parameters in real time further comprises:
in the idle stage, controlling the radio-frequency transmit chain to transmit a second radio-frequency pulse to the radio-frequency transmit coil;
during transmission of the second radio-frequency pulse, acquiring, in real time, a second radio-frequency power signal detected on the line between the radio-frequency transmit chain and the radio-frequency transmit coil; and
acquiring the scattering parameters on the basis of the second radio-frequency power signal.

3. The method according to claim 2, wherein each of the frequency of the second radio-frequency pulse and the frequency of the first radio-frequency pulse is an operating frequency of the magnetic resonance imaging system, the first radio-frequency pulse has a first power capable of exciting the object under detection, and the second radio-frequency pulse has a second power incapable of exciting the object under detection.

4. The method according to claim 1, wherein the magnetic resonance imaging system further comprises a first additional radio-frequency transmit chain, and the step of acquiring the scattering parameters in real time further comprises:
in the idle stage, controlling the first additional radio-frequency transmit chain to transmit a third radio-frequency pulse to the radio-frequency transmit coil;
during transmission of the third radio-frequency pulse, acquiring, in real time, a third radio-frequency power signal detected on a line between the first additional radio-frequency transmit chain and the radio-frequency transmit coil; and
acquiring the scattering parameters on the basis of the third radio-frequency power signal.

5. The method according to claim 4, wherein a frequency range of the third radio-frequency pulse deviates from an operating frequency range of the magnetic resonance imaging system.

6. The method according to claim 4, wherein a power of the third radio-frequency pulse is in milliwatts or watts.

7. The method according to claim 1, wherein the magnetic resonance imaging system further comprises a second additional radio-frequency transmit chain, and acquiring the scattering parameters in real time further comprises:
in the signal acquisition stage, controlling the second additional radio-frequency transmit chain to transmit a fourth radio-frequency pulse to the radio-frequency transmit coil;
during transmission of the fourth radio-frequency pulse, acquiring, in real time, a fourth radio-frequency power signal detected on a line between the second additional radio-frequency transmit chain and the radio-frequency transmit coil; and
acquiring the scattering parameters on the basis of the fourth radio-frequency power signal.

8. The method according to claim 7, wherein a frequency range of the fourth radio-frequency pulse deviates from an operating frequency range of the magnetic resonance imaging system.

9. The method according to claim 1, wherein the breathing information of the object under detection is acquired, on the basis of a first filter, from the scattering parameters acquired in real time, and the motion information of the object under detection is acquired, on the basis of a second filter, from the scattering parameters acquired in real time.

10. A magnetic resonance imaging method, comprising the breathing and motion monitoring method according to claim 1, further comprising: processing image data of the object under detection on the basis of at least one of the breathing information and the motion information of the object under detection.

11. A computer-readable storage medium, comprising a stored computer program, wherein the computer program, when being executed, implements the method according to claim 1.

12. A magnetic resonance imaging system, comprising:
a scanner, comprising a radio-frequency transmit chain and a radio-frequency transmit coil, an object under detection being positioned relative to the radio-frequency transmit coil,
a controller, configured to control the scanner to perform a scanning sequence on the object under detection to acquire image data of the object under detection, the scanning sequence comprising a radio-frequency excitation stage, a signal acquisition stage, and an idle stage between the radio-frequency excitation stage and the signal acquisition stage, wherein in the radio-frequency excitation stage, the radio-frequency transmit chain transmits a first radio-frequency pulse to the radio-frequency transmit coil; and,
a signal processor, configured to:
acquire scattering parameters in real time during the scanning sequence,
comprising: in the radio-frequency excitation stage, acquiring, in real time, a first radio-frequency power signal detected on a line between the radio-frequency transmit chain and the radio-frequency transmit coil, and acquiring the scattering parameters on the basis of the first radio-frequency power signal; and
acquire at least one of breathing information and motion information of the object under detection on the basis of the scattering parameters acquired in real time.

13. The system according to claim 12, wherein the controller is further configured to: in the idle stage, control the radio-frequency transmit chain to transmit a second radio-frequency pulse to the radio-frequency transmit coil; and
the signal processor is further configured to:
during transmission of the second radio-frequency pulse, acquire, in real time, a second radio-frequency power signal detected on the line between the radio-frequency transmit chain and the radio-frequency transmit coil; and
acquire the scattering parameters on the basis of the second radio-frequency power signal.

14. The system according to claim 13, wherein each of the frequency of the second radio-frequency pulse and the frequency of the first radio-frequency pulse is an operating frequency of the magnetic resonance imaging system, the first radio-frequency pulse has a first power capable of exciting the object under detection, and the second radio-frequency pulse has a second power incapable of exciting the object under detection.

15. The system according to claim 12, further comprising a first additional radio-frequency transmit chain, wherein the controller is further configured to: in the idle stage, control the first additional radio-frequency transmit chain to transmit a third radio-frequency pulse to the radio-frequency transmit coil; and
the signal processor is further configured to:
during transmission of the third radio-frequency pulse, acquire, in real time, a third radio-frequency power signal detected on a line between the first additional radio-frequency transmit chain and the radio-frequency transmit coil; and
acquire the scattering parameters on the basis of the third radio-frequency power signal.

16. The system according to claim 15, wherein a frequency range of the third radio-frequency pulse deviates from an operating frequency range of the magnetic resonance imaging system.

17. The system according to claim 15, wherein a power of the third radio-frequency pulse is in milliwatts or watts.

18. The system according to claim 12, further comprising a second additional radio-frequency transmit chain, wherein the controller is further configured to: in the signal acquisition stage, control the second additional radio-frequency transmit chain to transmit a fourth radio-frequency pulse to the radio-frequency transmit coil; and
the signal processor is further configured to:
during transmission of the fourth radio-frequency pulse, acquire, in real time, a fourth radio-frequency power signal detected on a line between the second additional radio-frequency transmit chain and the radio-frequency transmit coil; and
acquire the scattering parameters on the basis of the fourth radio-frequency power signal.

19. The system according to claim 18, wherein a frequency range of the fourth radio-frequency pulse deviates from an operating frequency range of the magnetic resonance imaging system.

20. The system according to claim 12, wherein the signal processor is configured to extract the breathing information of the object under detection from the scattering parameters acquired in real time on the basis of a first filter, and the signal processor is configured to extract the motion information of the object under detection from the scattering parameters acquired in real time on the basis of a second filter.

21. The system according to claim 12, further comprising an image data processor configured to process image data of the object under detection on the basis of at least one of the breathing information and the motion information of the object under detection.
